(19) United States
(12) Patent Application Publication
(10) Pub. No.: US 2026/0365595 A1
(43) Pub. Date: Nov. 19, 2026

(54) A PROCESS FOR SIMULTANEOUSLY CONVERTING HYDROGEN HALIDES TO HALOGENS ANDCARBON TO HYDROCARBONS

(71) Applicants: Ahmad DEHESTANI; Eric W. MCFARLAND, Santa Barbara, CA (US)

(72) Inventors: Ahmad DEHESTANI; Eric W. MCFARLAND, Santa Barbara, CA (US)

(21) Appl. No.: 19/141,245

(22) PCT Filed: Dec. 22, 2023
(86) PCT No.: PCT/US2023/085637

§ 371 Date: Jun. 19, 2025

Related U.S. Application Data
(60) Provisional application No. 63/477,309, filed on Dec. 27, 2022.

(51) Int. Cl. C07C 1/32; C25B 1/26

(52) U.S. Cl. CPC C07C 1/328; C25B 1/26

(57) ABSTRACT

A process for producing hydrocarbons and halogens includes contacting solid carbon with a reactive metal to produce a metal carbide, reacting the metal carbide with a hydrogen halide to produce a product comprising a hydrocarbon and a metal halide, and converting the metal halide to an elemental metal and an elemental halogen.

### CROSS-REFERENCE TO RELATED APPLICATIONS

This application claims priority to U.S. Provisional Application No. 63/477,309 filed on Dec. 27, 2022 and entitled “PROCESS FOR SIMULTANEOUSLY CONVERTING HYDROGEN HALIDES TO HALOGENS AND CARBON TO HYDROCARBONS”, the entire disclosure of which is incorporated herein by reference.

### BACKGROUND

The modern petrochemical industry makes extensive use of cracking and fractionation technology to produce and separate various desirable compounds from crude oil. Cracking and fractionation operations are energy intensive and generate considerable quantities of greenhouse gases. Production of valuable chemicals starting with other feedstocks would be useful.

### SUMMARY

In some embodiments, a process for producing hydrocarbons and halogens comprises: contacting solid carbon with a reactive metal to produce a metal carbide, reacting the metal carbide with a hydrogen halide to produce a product comprising a hydrocarbon and a metal halide, and converting the metal halide to an elemental metal and an elemental halogen.

In some embodiments, a process for producing hydrocarbons and halogens comprises contacting a carbon oxide with a reactive metal to produce a metal carbide and a reactive metal oxide, reacting the metal carbide with a first portion of a hydrogen halide to produce a product comprising a hydrocarbon and a first portion of a metal halide, reacting the reactive metal oxide with a second portion of the hydrogen halide to produce a second portion of the metal halide and water, and converting the first portion of the metal halide and the second portion of the metal halide to an elemental metal and an elemental halogen.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description:

FIG. 1 schematically illustrates an integrated continuous process.

FIG. 2 schematically illustrates a reactor for the integrated production of a metal carbide and acetylene.

FIG. 3 schematically illustrates an integrated continuous process for producing chlorine and acetylene from carbon and HCl.

FIG. 4 schematically illustrates an integrated continuous process for converting lithium carbonate and HCl to chlorine and acetylene and lithium.

FIG. 5 schematically illustrates how carbon dioxide can be captured and converted with HCl to chlorine and acetylene.

FIG. 6 schematically illustrates a mixed feed stream of HCl and oxides of lithium to produce chlorine and lithium.

### DEFINITIONS

Reactive metal: Refers to a metal that reacts with carbon to form a carbide.

Reactant: Any substance that enters into and is potentially altered in the course of a chemical transformation.

Product: A substance resulting from a set of conditions in a chemical or physical transformation.

Reactor: A container or apparatus in which substances are made to undergo chemical transformations.

Halogen: Oxidant molecule from the group including bromine, chlorine, iodine, fluorine.

### DETAILED DESCRIPTION

The processes, systems, and methods disclosed herein demonstrate how molecular halogens and hydrocarbons can be produced from a solid carbon source and hydrogen halides. Generally, carbon oxidation generates undesirable carbon oxides, however, reactions of carbon with certain metals to form stable metal carbides results provides a reaction pathway which allows partial oxidation of the carbon to form hydrocarbons and metal salts. Electrolysis of the salts is used to regenerate the active metal and molecular halogens. Chlorine is among the most produced inorganic chemicals in the world. It is an essential intermediate for various chemical products including polyvinyl chloride. Cl₂ is primarily produced by electrolysis requiring large amounts of electricity, generally with the co-production of low value hydrogen and sodium hydroxide. Often when chlorine is utilized, hydrogen chloride (HCl) is a co-produced byproduct requiring a recycling strategy. Electrochemical recovery of chlorine from HCl can be done commercially. A gas phase process for HCl conversion to chlorine using oxygen has been developed (the Deacon Process) but not widely deployed for practical reasons. There are large streams of HCl where improved chlorine recovery processes would be beneficial. Acetylene is a key intermediate of the chemical industry and an important industrial gas. Today, it is synthesized by three main pathways, 1) hydrolysis of calcium carbide from coal and calcium oxide, 2) partial oxidation of natural gas, and 3) electric arc pyrolysis. All processes today are energy intensive and co-produce carbon dioxide.

In the calcium carbide process, calcium carbide is produced in abundance from coal, by reacting CaO with carbon. The resulting carbide can be reacted with water to produce acetylene and calcium hydroxide as shown in the following equations:

3C+CaO→CaC₂+CO

CaC₂+2H₂⇄2C₂H₂+Ca(OH)₂

The processes and systems disclosed herein make use of the reactivity of selected metals with carbon to form reactive metal carbides, which can be reacted with hydrogen halides instead of water to form metal halide salts and hydrocarbons. The realization that the carbide formation can be coupled with a subsequent reaction with a hydrogen halide is an important aspect of the process. Historically metal carbides have been grouped in terms of the gas evolved upon hydrolysis (methane, acetylene, etc).

TABLE 1

| Carbide Name | Carbide-Anion | Examples | Gas evolved |
|---|---|---|---|
| Methanides | C⁴⁻ | Al₄C₃, Mg₂C, Be₂C | CH₄ |
| Acetylides | C₂²⁻ | CaC₂, LaC₂, Li₂C₂ | C₂H₂ |
| Sesquicarbides | C₃⁴⁻ | Li₄C₃, Mg₂C₃ | CH₃CCH, |
| | | | CH₂CCH₂ |

In some embodiments, a process and system for carrying out the process as described herein can be used to produce hydrocarbons and halogens from one or more feedstocks containing hydrogen halides and carbon. In the process, metal carbides can be used as a reactant. In an optional step, carbon (e.g., solid carbon, etc.) can be contacted with a reactive metal under conditions suitable to react the carbon with the reactive metal to produce the metal carbides. The metal carbides can then be reacted with a hydrogen halide under suitable conditions to produce a hydrocarbon product and a corresponding metal halide. The reactive metal can be regenerated from the metal halide using a suitable process to produce a halogen product (e.g., a corresponding element halogen, etc.).

As an example of this process, acetylene can be produced by contacting solid carbon with a reactive metal to form an acetylide. Any suitably reactive metal can be used. In some aspects, the reactive metal can include, but is not limited to, sodium, lithium, potassium, lanthanum, calcium, magnesium, or any mixture thereof. In order to avoid the presence of oxygen in the system, the solid carbon can initially be processed to remove any oxygen present in the solid carbon.

While any suitable reaction system can be used, a molten system can be used in some aspects. In this system, the reactive metal and/or a salt of the reactive metal can be maintained as a molten phase above its melting temperature, and the carbon can be contacted with the molten phase to form the metal carbide. For example, the reaction between the carbon and the molten phase can occur at a temperature between about 30 °C and about 800 °C at a pressure between about 0.1 bar and about 10 bar.

Upon reaction of the carbon with the reactive metal in the molten phase to form the metal carbide, a two-phase mixture can be formed. The first phase can be the molten phase while the second phase can include a separated metal carbide phase comprising the metal carbide, which can be at least partially insoluble in the molten phase. The resulting phase separation can result in the formation of a two-phase mixture that can stratify based on a density difference between the phases.

The metal carbide can then be contacted with a hydrogen halide. In the example of producing acetylene, the acetylide can then be contacted with a hydrogen halide to produce acetylene and a corresponding metal halide. In some aspects, the halogen in the hydrogen halide can be iodine, bromine, chlorine, or any combination thereof. The hydrogen halide can be contacted with the metal carbide at a temperature between about −20 °C and about 800 °C, or at a temperature between about 30 °C and about 800 °C. The hydrogen halide can be contacted with the metal carbide at a pressure of between about 0.1 bar and about 30 bar.

In the event that any metal oxides are formed due to presence of water, oxygen, or impurities in the reactive metal and/or in the metal carbide reactor, the metal oxides can be removed by reaction with the hydrogen halides to form water and the metal halide. In order to help avoid the presence of oxygen, the hydrogen halide used in the reaction can be passed through a dryer to first dry the hydrogen halide prior to reacting the hydrogen halide with the metal carbide.

The acetylene can be taken as a product stream in the gas phase. The gas phase hydrocarbon product can be cooled to stabilize the hydrocarbon. In some embodiments, the hydrocarbon product can be passed through a direct contact cooler to quench the hydrocarbon. For example, the hydrocarbon can pass through a hydrocarbon oil or water to cool the hydrocarbon product. The heat removed from the hydrocarbon product can be used to heat an endothermic process such as the electrolysis of the metal halide to form the reactive metal and the halogen gas. When water is used to cool the hydrocarbon product, the hydrocarbon product can then pass through a dryer to remove any water within the hydrocarbon product.

The metal halide can be present as a molten halide salt that can be phase separated from the hydrocarbon product (e.g., the acetylene). The molten halide salt can be converted back to the reactive metal and the corresponding elemental halide using a suitable process. For example, the metal halide an be decomposed using an electrochemical cell to produce a halogen gas and the reactive metal. The process can then be repeated by recycling the reactive metal to the metal carbide formation reactor. The halogen can be removed from the system for further uses and/or used in a process to generate hydrogen halide for further use in the process. Depending on the downstream use of the halogen, the halogen product can pass through a purification process to produce high purity halogen for use in other processes.

The processes described herein can be carrier out in a number of reactor systems. In some aspects, any suitable reactors can be used to perform the individual steps and/or the steps can be combined to produce the overall reactions with the production of a halogen and a hydrocarbon product based on the input of hydrogen halide and solid carbon. The reactors and vessels may use appropriate materials of construction for use under the conditions used to carry out the reactions.

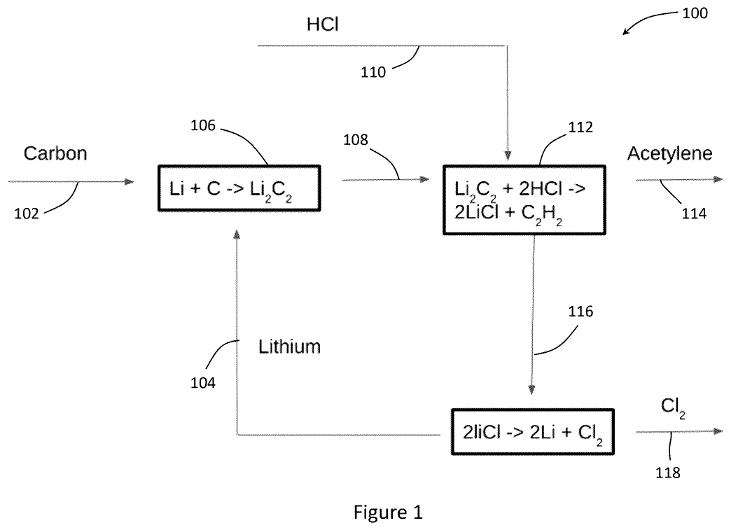

An embodiment of a system and process 100 to produce acetylene is shown schematically in FIG. 1. The process 100 is illustrated with the use of lithium as the reactive metal and chlorine as the halogen as examples only, and any of the reactive metals and halogens disclosed herein could be used. As shown, the process 100 can use lithium metal to react with a carbon feedstock introduced in stream 102 within a reaction process 106 to form lithium carbide (e.g., Li₂C₂). The Li₂C₂ can then be passed as stream 108 to a second reaction process 112 and reacted with the second feedstock, HCl, in stream 110 to form acetylene (the reaction having a ΔH_{850C}=−300 KJ/mole ΔG_{850C}=−232 KJ/mole), which can leave as stream 114, and LiCl, which can pass to a recover process as stream 116. The LiCl₂ can then be electrolytically decomposed to produce chlorine in stream 118 and regenerate the lithium metal (the reaction having a ΔH_{850C}=383 KJ/mole ΔG_{850C}=323 KJ/mole), which can be recycled to the first reaction process 106 as stream 104. The process is shown in the following equations:

2C+2Li→C₂Li₂

C₂Li₂+2HCl→C₂H₂+2LiCl

2LiCl→2Li+Cl₂

Unique features of the process include high selectivity to acetylene and no co-generation of carbon dioxide, which are significant advances over prior art. Further, there is no need to generate hydroxides which may or may not have end uses, and there are no waste-water challenges. This is the first process to make use of the reaction of hydrogen halides with acetylides to produce hydrocarbons. The carbon sources are many and may make use of hydrogen produced in hydrocarbon pyrolysis processes and carbons containing impurities including alkali halides. The process can be readily integrated into production facilities making use of chlorine and producing HCl, and the process offers a low cost means of chlorine recovery from HCl.

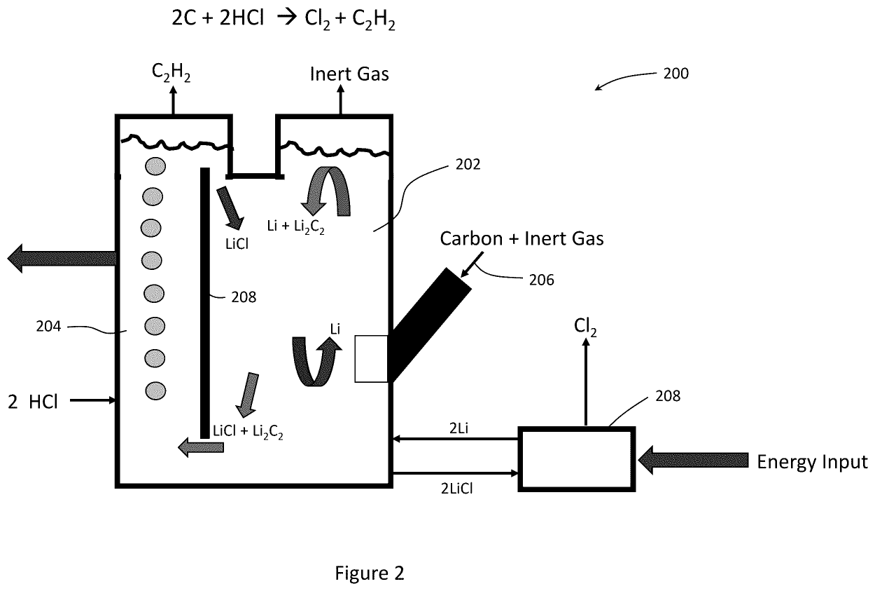

In some embodiments, a reactor 200 as shown in FIG. 2 can be utilized whereby the density difference between the molten reactive metal 202 (e.g., a molten alkali metal such as Li, Na, etc.) and its molten salt 204 allow for stratification based on density within the reactor 200 of the metal 202 and the salt 204. Carbon can be introduced continuously in stream 206 into the molten metal 202 to react with the metal and produce the denser carbide product, which can phase separate to the salt layer and travel laterally to a bubble lift column. The bubble lift column can be formed as a separate reactor fluidly connected to the main reactor 200 or positioned as a portion of the reactor 200. An internal wall of baffle 208 can be used to direct the flow of the molten phase within the reactor 200. Hydrogen halide can be introduced in stream 210 and react with the metal carbide to produce a hydrocarbon product such as acetylene and the alkali salt (e.g., LiCl, etc.), where the liquids can circulate based on the bubble lift drive provided by the rising gas. The electrochemical cell 208 where the alkali metal can be regenerated can receive the salt from the reactor 200, generate the molten metal, return the molten metal to the reactor, and generate gas phase chlorine. This embodiment allows the reactor 200 to include one or two vessels for carrying out the carbon and reactive metal reactions, and the reaction of the metal carbide with the hydrogen halide while taking advantage of the solubility and density differences of the components to generate a circulation.

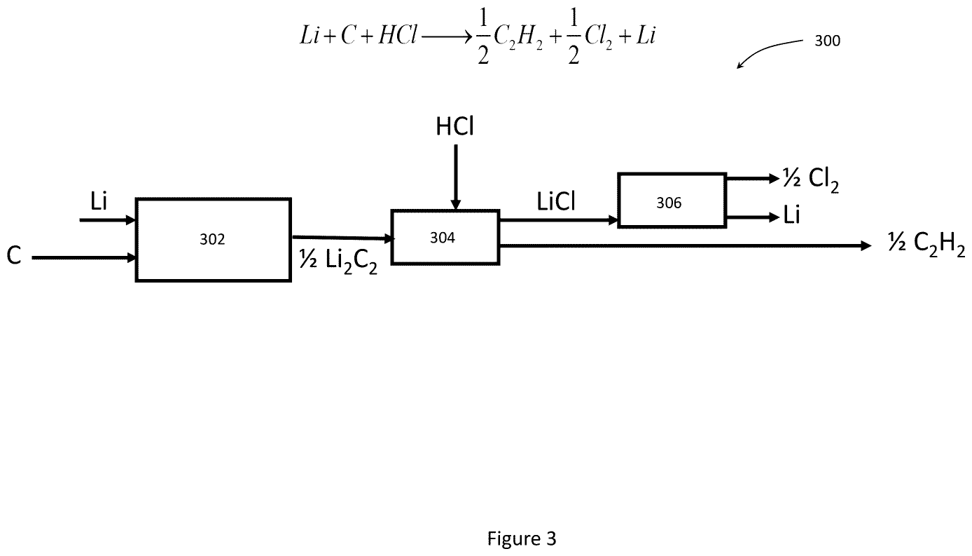

FIG. 3 illustrates the overall process using lithium and chlorine as exemplary materials. As shown, the overall reaction can be expressed as:

$$Li ιC \prod HCl \times λ \frac{1}{2} {C}_{2} {H}_{1} 1 \frac{1}{2} {Cl}_{2} 2 Li$$

The reaction can be separated into one or more sub-reaction steps, including a first step 302 of reacting the reactive metal with solid carbon. As illustrated, the reactive metal can comprise lithium. The resulting metal carbide (e.g., lithium carbide) can then be contacted with a hydrogen halide in a second step 304. As illustrated, the lithium carbide can be contacted and reacted with hydrogen chloride to produce a hydrocarbon product (e.g., acetylene) and a metal halide such as lithium chloride. The metal halide can then pass to a third step 306 where the reactive metal and the halogen can be regenerated. As shown, this could include regenerating the lithium metal and chlorine. While FIG. 3 uses lithium and chlorine as examples, any of the reactive metals and halides described herein can also be used.

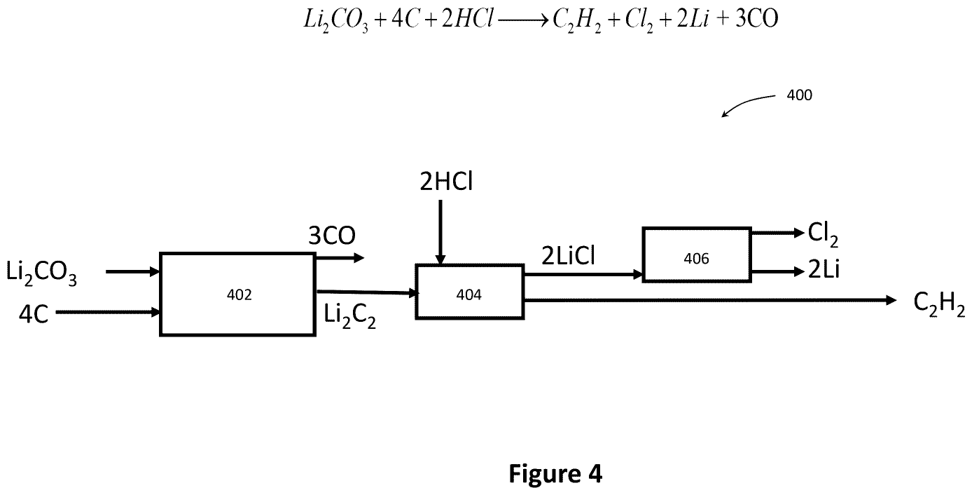

In some embodiments, a reactive metal salt can be used as a starting material in addition to or in place of a reactive metal. FIG. 4 illustrates an overall process 400 using a reactive metal salt in the process. Similar to FIG. 3, lithium carbonate and chlorine are illustrated as the starting materials, but other reactive metal salts and halogens can be used. For example, the reactive metal salt can include one or more salts of any of the reactive metals disclosed herein.

As shown in FIG. 4, the overall reaction can be expressed as:

Li₂CO₃4C2HCl× [symbol] C₂H₂2Cl₂X2Li+3CO

As shown, lithium carbonate can be reacted together with carbon in a first reaction 402 to produce the alkali carbide and carbon monoxide. The carbon monoxide can be removed from the system. The resulting metal carbide can then be processed as described with respect to FIG. 3. For example, the carbide is contacted with hydrogen halide in step 404 to form the acetylene product and the alkali salt, which is further processed by electrolysis in step 405. The final products can comprise Li metal, acetylene, chlorine, and carbon monoxide. FIG. 4 illustrates the use of a reactive metal salt for forming the metal carbide used in to contact the hydrogen halide.

In some embodiments, carbon dioxide can be captured and reacted with a reactive metal (e.g., lithium, etc.) to produce the alkali carbide and a metal oxide (e.g., lithium oxide, etc.). The overall reaction can be expressed as:

2CO₂,210Liι10HClλ [symbol] CH₂H₂25Cl₂10Li X4H₂O

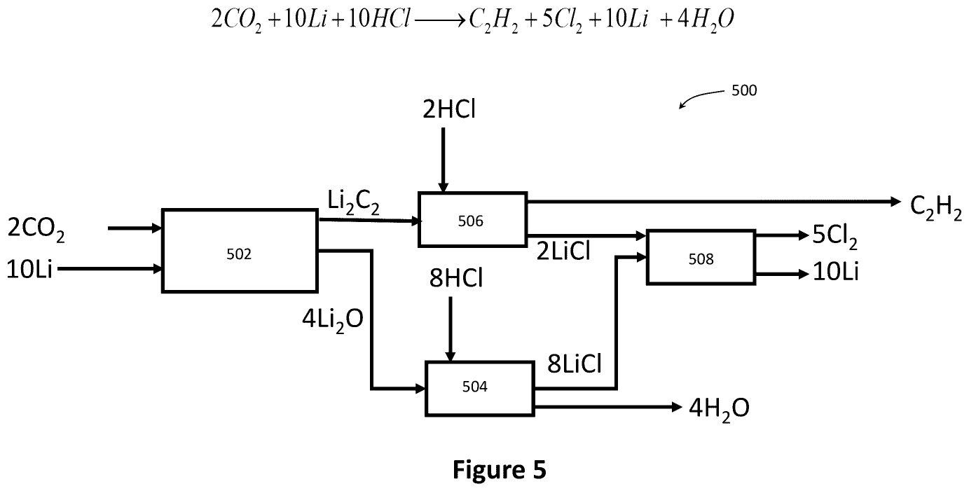

The process is shown schematically in FIG. 5. Initially, carbon dioxide can be contacted with the reactive metal in step 502 to form a metal carbide and a reactive metal oxide. The carbide can be contacted with hydrogen halide in step 506 to form the hydrocarbon (e.g., acetylene, etc.) product and the alkali salt, which can be further processed in step 508 by electrolysis. The reactive metal oxide can be contacted with hydrogen halide in step 504 to produce the metal halide and water, where the metal halide can be further processed in step 508. The ability to process the metal oxide with the hydrogen halide can allow the carbon dioxide to be converted into water and removed from the system. The final products can comprise the hydrocarbon product (e.g., acetylene), the halogen (e.g., chlorine), and water.

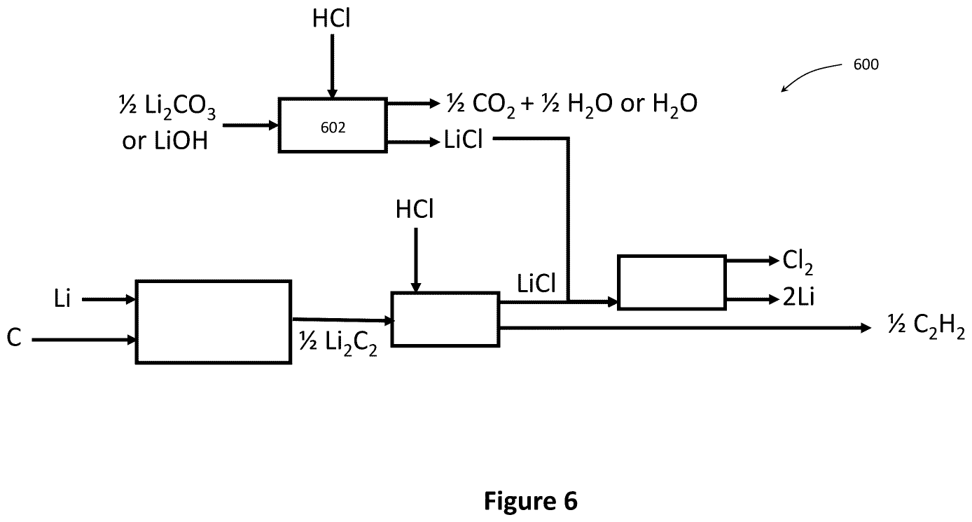

Mixtures of different reactive metal forms may also be processed using the hydrogen halides and combined in the process for different mixtures of products as illustrated in FIG. 6. For example, the process 600 of FIG. 6 can use a step in which a reactive metal compound comprising oxygen can be contacted with a hydrogen halide to produce a metal halide and remove the oxygens as one or more oxygen containing compounds. In the example shown, lithium carbonate and/or lithium hydroxide can be converted into lithium chloride and carbon dioxide and water. The carbon dioxide and water can be separated from the metal halide. The remaining reactions can be the same or similar to those described herein, where the resulting metal halide can be processed along with any metal halide generated in the remainder of the system.

As disclosed herein, various systems can be used to convert compounds including carbon into a hydrocarbon product using a reactive metal with a hydrogen halide intermediate. Additional oxides can be processed within the system to generate a hydrocarbon product while allowing the oxygen to leave the system as water in some embodiments.

### EXAMPLES

The disclosure having been generally described, the following examples are given as particular embodiments of the disclosure and to demonstrate the practice and advantages thereof. It is understood that the examples are given by way of illustration and are not intended to limit the specification or the claims in any manner.

### Example 1

### Generation of Acetylene from HCl and Li₂C₂

In one example, Li₂C₂ is produced in a 500 ml stainless reactor maintained at 850 °C by contacting carbon (acetylene black) with molten Li in an Argon atmosphere. After reaction the vessel is cooled to 200 °C and the remaining molten Li is decanted from the solid Li₂C₂. The solid is transferred in a glove box to a quartz reaction vessel and cooled to room temperature. The quartz reaction vessel is then evacuated and cooled further to 0 °C in an ice bath. HCl vapor was slowly introduced to 0.1 atm while controlling the temperature and monitoring the vessel pressure, which is allowed to increase to 1 atm. After 30 minutes the gas contents are sampled by bubbling through a chilled water bath. Acetylene is measures as the only gas phase product.

Having described various systems and processes, certain aspects can include, but are not limited to:

A first aspect can include a process for producing hydrocarbons and halogens comprising: contacting solid carbon with a reactive metal to produce a metal carbide; reacting the metal carbide with a hydrogen halide to produce a product comprising a hydrocarbon and a metal halide; and converting the metal halide to an elemental metal and an elemental halogen.

A second aspect can include a process for producing hydrocarbons and halogens comprising: contacting a carbon oxide with a reactive metal to produce a metal carbide and a reactive metal oxide; reacting the metal carbide with a first portion of a hydrogen halide to produce a product comprising a hydrocarbon and a first portion of a metal halide; reacting the reactive metal oxide with a second portion of the hydrogen halide to produce a second portion of the metal halide and water; and converting the first portion of the metal halide and the second portion of the metal halide to an elemental metal and an elemental halogen.

A third aspect can include the process of the first or second aspect, wherein the metal carbide comprises an acetylide, and wherein the hydrocarbon comprises acetylene.

A fourth aspect can include the process of any one of the first to third aspects, wherein the reactive metal comprises sodium, lithium, lanthanum, calcium, or any combination thereof.

A fourth aspect can include the process of any one of the first to fourth aspects, wherein the halogen comprises chlorine, bromine, iodine, or any combination thereof.

A sixth aspect can include the process of any one of the first to fifth aspects, wherein converting the metal halide to the elemental metal and the elemental halogen comprises using electrochemical decomposition to convert the metal halide to the elemental metal and the elemental halogen.

A seventh aspect can include the process of any one of the first to sixth aspects, wherein contacting the solid carbon with the reactive metal comprises: contacting the solid carbon with the reactive metal in a molten phase.

An eighth aspect can include the process of the seventh aspect, further comprising: separating the metal carbide from the molten phase; and contacting the hydrogen halide in a gas phase with the metal carbine to form the hydrocarbon and the metal halide as a molten salt.

A ninth aspect can include the process of the eighth aspect, wherein separating the metal carbide from the molten phase uses a density difference, a solubility difference, or both of the metal carbide and the molten phase.

A tenth aspect can include the process of any one of the first to ninth aspects, further comprising: cooling the hydrocarbon using a direct contact cooler.

An eleventh aspect can include the process of any one of the first to tenth aspects, wherein the reactive metal comprises lithium, sodium, potassium, calcium, magnesium, lanthanum, or any combination thereof.

A twelfth aspect can include the process of any one of the first to eleventh aspects, wherein the contacting the solid carbon and the reactive metal occurs at a temperature between 30 °C and 800 °C and a pressure of between 0.1 and 10 bar.

A thirteenth aspect can include the process of any one of the first to twelfth aspects, wherein the contacting the solid carbon and the reactive metal occurs in a molten mixture of the reactive metal and a reactive metal salt.

A fourteenth aspect can include the process of any one of the first to sixth aspects, wherein reacting the metal carbide with the hydrogen halide occurs at a temperature between 30 °C and 800 °C and a pressure of between 0.1 and 30 bar.

A fifteenth aspect can include the process of any one of the first to thirteenth aspects, where reacting the metal carbide with the hydrogen halide occurs at a temperature between −20 °C and 800 °C.

A sixteenth aspect can include the process of any one of the first to fifteenth aspects, further comprising: drying the hydrogen halide prior to reacting the metal carbide with the hydrogen halide.

A seventeenth aspect can include the process of any one of the first to sixteenth aspects, further comprising: removing substantially all of the oxygen from the solid carbon prior to contacting the solid carbon with the reactive metal.

An eighteenth aspect can include the process of any one of the first to seventeenth aspects, further comprising: purifying the elemental halogen.

A nineteenth aspect can include the process of any one of the first to eighteenth aspects, further comprising: drying the hydrocarbon.

A twentieth aspect can include the process of any one of the first to nineteenth aspects, wherein the reactive metal is a reactive metal salt, wherein contacting the solid carbon with the reactive metal salt produces the metal carbide and a carbon oxide.

A twenty first aspect can include the process of the twentieth aspect, wherein the reactive metal salt comprises a reactive metal carbonate.

A twenty second aspect can include the process of any one of the first to twenty first aspects, further comprising: contacting a compound comprising the reactive metal and oxygen with a portion of the hydrogen halide to produce a portion of the metal halide and a stream comprising one or more compounds comprising the oxygen.

It is to be further understood that the present description is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present systems and methods. It must be noted that as used herein and in the appended claims (in this application, or any derived applications thereof), the singular forms “a,” “an,” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this description belongs. Preferred methods, techniques, devices, and materials are described, although any methods, techniques, devices, or materials similar or equivalent to those described herein may be used in the practice or testing of the present systems and methods. Structures described herein are to be understood also to refer to functional equivalents of such structures. The present systems and methods will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims may be formulated in this Application or of any further Application derived therefrom, to particular combinations of features, it should be understood that the scope of the disclosure also includes any novel feature or any novel combination of features disclosed herein either explicitly or implicitly or any generalization thereof, whether or not it relates to the same systems or methods as presently claimed in any Claim and whether or not it mitigates any or all of the same technical problems as do the present systems and methods.

Features which are described in the context of separate embodiments may also be provided in combination in a single embodiment. Conversely, various features which are, for brevity, described in the context of a single embodiment, may also be provided separately or in any suitable sub-combination. The Applicants hereby give notice that new claims may be formulated to such features and/or combinations of such features during the prosecution of the present Application or of any further Application derived therefrom.

1. A process for producing hydrocarbons and halogens, the process comprising:
contacting solid carbon with a reactive metal to produce a metal carbide;
reacting the metal carbide with a hydrogen halide to produce a product comprising a hydrocarbon and a metal halide; and
converting the metal halide to an elemental metal and an elemental halogen.

2. (canceled)

3. The process of claim 1, wherein the metal carbide comprises an acetylide, and wherein the hydrocarbon comprises acetylene.

4. The process of claim 1, wherein the reactive metal comprises sodium, lithium, lanthanum, calcium, potassium, magnesium or any combination thereof.

5. The process of claim 1, wherein the halogen comprises chlorine, bromine, iodine, or any combination thereof.

6. The process of claim 1, wherein converting the metal halide to the elemental metal and the elemental halogen comprises using electrochemical decomposition to convert the metal halide to the elemental metal and the elemental halogen.

7. The process of claim 1, wherein contacting the solid carbon with the reactive metal comprises:
contacting the solid carbon with the reactive metal in a molten phase.

8. The process of claim 7, further comprising:
separating the metal carbide from the molten phase; and
contacting the hydrogen halide in a gas phase with the metal carbine to form the hydrocarbon and the metal halide as a molten salt.

9.-11. (canceled)

12. The process of claim 1, wherein the contacting the solid carbon and the reactive metal occurs at a temperature between 30 °C and 800 °C and a pressure of between 0.1 and 10 bar, and where reacting the metal carbide with the hydrogen halide occurs at a temperature between 30 °C and 800 °C and a pressure of between 0.1 and 30 bar.

13. The process of claim 1, wherein the contacting the solid carbon and the reactive metal occurs in a molten mixture of the reactive metal and a reactive metal salt.

14.-15. (canceled)

16. The process of claim 1, further comprising:
drying the hydrogen halide prior to reacting the metal carbide with the hydrogen halide.

17.-19. (canceled)

20. The process of claim 1, wherein the reactive metal is a reactive metal salt, wherein contacting the solid carbon with the reactive metal salt produces the metal carbide and a carbon oxide.

21. The process of claim 20, wherein the reactive metal salt comprises a reactive metal carbonate.

22. (canceled)

23. A process for producing hydrocarbons and halogens, the process comprising:
contacting a carbon oxide with a reactive metal to produce a metal carbide and a reactive metal oxide;
reacting the metal carbide with a first portion of a hydrogen halide to produce a product comprising a hydrocarbon and a first portion of a metal halide;
reacting the reactive metal oxide with a second portion of the hydrogen halide to produce a second portion of the metal halide and water;
converting the first portion of the metal halide and the second portion of the metal halide to an elemental metal and an elemental halogen.

24. The process of claim 23, wherein the metal carbide comprises an acetylide, and wherein the hydrocarbon comprises acetylene.

25. The process of claim 23, wherein the reactive metal comprises sodium, lithium, lanthanum, potassium, calcium, magnesium, or any combination thereof.

26. The process of claim 23, wherein the elemental halogen comprises chlorine, bromine, iodine, or any combination thereof.

27. The process of claim 23, wherein converting the first portion of the metal halide and the second portion of the metal halide to the elemental metal and the elemental halogen comprises using electrochemical decomposition to convert the metal halide to the elemental metal and the elemental halogen.

28. The process of claim 23, wherein the contacting the carbon oxide and the reactive metal occurs at a temperature between 30 °C and 800 °C and a pressure of between 0.1 and 10 bar, and where reacting the metal carbide with the first portion of the hydrogen halide occurs at a temperature between 30 °C and 800 °C and a pressure of between 0.1 and 30 bar.

29. The process of claim 23, wherein the contacting the carbon oxide and the reactive metal occurs in a molten mixture of the reactive metal and a reactive metal salt.

30. The process of claim 23, wherein the reactive metal is a reactive metal salt, wherein contacting the solid carbon with the reactive metal salt produces the metal carbide and a carbon oxide.

31. The process of claim 30, wherein the reactive metal salt comprises a reactive metal carbonate.
